(19) United States
(12) Patent Application Publication
(10) Pub. No.: US 2010/0311609 A1
(43) Pub. Date: Dec. 9, 2010

(54) Methylation Profile of Neuroinflammatory Demyelinating Diseases

(75) Inventors: Anatoliy A. Melnikov, Glenview, IL (US); Victor V. Levenson, Chicago, IL (US)

Correspondence Address: ANDRUS, SCEALES, STARKE & SAWALL, LLP, 100 EAST WISCONSIN AVENUE, SUITE 1100, MILWAUKEE, WI, 53202, US

(73) Assignee: NORTHWESTERN UNIVERSITY, Evanston, IL (US)

(21) Appl. No.: 12/856,941

(22) Filed: Aug. 16, 2010

Related U.S. Application Data
(63) Continuation of application No. 12/013,842, filed on Jan. 14, 2008.
(60) Provisional application No. 60/880,130, filed on Jan. 12, 2007.

(51) Int. Cl. C40B 30/04; C12Q 1/68

(52) U.S. Cl. 506/9; 435/6

(57) ABSTRACT

The present invention relates to compositions and methods for diagnosing neuroinflammatory demyelinating diseases, including but not limited to, multiple sclerosis. In particular, the present invention provides methods of identifying methylation patterns in genes associated with neuroinflammatory demyelinating diseases.

### CROSS-REFERENCE TO RELATED APPLICATIONS

The present application claims the benefit under 35 U.S.C. §119(e) to U.S. provisional patent application No. 60/880,130, filed on Jan. 12, 2007, the entire content of which is incorporated herein by reference.

### FIELD

The present invention relates to compositions and methods for diagnosing neuroinflammatory demyelinating diseases, including but not limited to, multiple sclerosis. In particular, the present invention provides methods of identifying methylation patterns in genes associated with neuroinflammatory demyelinating diseases.

### BACKGROUND

A neuroinflammatory demyelinating disease is any disease of the nervous system in which the myelin sheath of neurons is damaged and inflammation occurs, thereby impairing the conduction of signals in the affected nerves, causing impairment in sensation, movement, cognition, or other functions depending on which nerves are involved. The term describes the effect of the disease, rather than its cause. Some demyelinating diseases are caused by infectious agents, some by autoimmune reactions, and some by unknown factors. Organo-phosphates, a class of chemicals that are the active ingredients in commercial insecticides such as sheep dip, weed-killers, and flea treatment preparations for pets, etc, will also demyelinate nerves. Demyelinating diseases include multiple sclerosis (MS), transverse myelitis, Guillain-Barré syndrome, and progressive multifocal leukoencephalopathy (PML).

Multiple sclerosis is difficult to diagnose in its early stages. In fact, definite diagnosis of MS cannot be made until there is evidence of at least two anatomically separate demyelinating events occurring at least thirty days apart. The McDonald criteria represent international efforts to standardize the diagnosis of MS using clinical data, laboratory data, and radiologic data.

Magnetic resonance imaging (MRI) of the brain and spine is often used to evaluate individuals with suspected MS. MRI shows areas of demyelination as bright lesions on T2-weighted images or FLAIR (fluid attenuated inversion recovery) sequences. Gadolinium contrast is used to demonstrate active plaques on T1-weighted images. Because MRI can reveal lesions that occurred previously, but produced no clinical symptoms, it can provide the evidence of chronicity needed for a definite diagnosis of MS. Testing of cerebrospinal fluid (CSF) can provide evidence of chronic inflammation of the central nervous system. The CSF is tested for oligoclonal bands, which are immunoglobulins found in 85% to 95% of people with definite MS. Combined with MRI and clinical data, the presence of oligoclonal bands can help make a definite diagnosis of MS.

The brain of a person with MS often responds less actively to stimulation of the optic nerve and sensory nerves. These brain responses can be examined using Visual evoked potentials (VEP) and somatosensory evoked potentials (SEP). Decreased activity on either test can reveal demyelination that may be otherwise asymptomatic. Along with other data, these exams can help find the widespread nerve involvement required for a definite diagnosis of MS. Future tests involving the measurement of anti-myelin proteins (e.g., myelin oligodendrocyte glycoprotein, myelin basic protein) may also have diagnostic potential, but to date there is no established role for these tests in diagnosing MS.

The signs and symptoms of MS can be similar to other medical problems, such as stroke, brain inflammation, infections such as Lyme disease (which can produce identical MRI lesions and CSF abnormalities), tumors, and other autoimmune problems, such as lupus. Therefore, straightforward testing for MS is complicated and a variety of different tests need to be applied to define the diagnosis.

Early detection of neuroinflammatory demyelinating diseases can hardly be underestimated. Early diagnosis has profound effects on survival rate, quality of life, and overall cost to society, so screening for these debilitating and oftentimes deadly diseases (especially with regards to PML) provides a valuable opportunity to promote a shift in diagnosis to early onset of these diseases, thereby causing earlier applied treatment regimens, better quality of life, and in some instances increased survival.

Thus, there is a need in the art for reliable diagnostic (e.g., detection) and prognostic methods to identify and monitor neuroinflammatory demyelinating diseases.

### SUMMARY

The present invention relates to compositions and methods for diagnosing neuroinflammatory demyelinating diseases, including but not limited to, multiple sclerosis. In particular, the present invention provides methods of identifying methylation patterns in genes associated with neuroinflammatory demyelinating diseases.

DNA methylation is one of the mechanisms for regulating gene expression. In abnormal cells, anomalous hypermethylation correlates with inactivation of tumor suppressors, while irregular hypomethylation correlates with activation of oncogenes. Changes of methylation change susceptibility of genomic DNA to methylation-sensitive restriction enzymes such that only hypomethylated DNA can be destroyed by such enzymes. Digestion with methylation-sensitive restriction enzymes leads to destruction of the integrity of the genomic DNA, such that it can no longer serve as a template for polymerase chain reaction (PCR) amplification; hypermethylated DNA is insensitive to methylation-sensitive restriction enzymes and can be amplified. The comparison of amplification products of undigested (control) and digested (test) DNA identifies hypo- and hypermethylated fragments. The technique involves (1) successful digestion of susceptible DNA with methylation-sensitive restriction enzymes, (2) amplification of selected fragments in control and test samples; (3) competitive hybridization of amplified products to a microarray (e.g., allowing for high-throughput analysis); and (4) scoring the results. Disease specific cell-free DNA is present in blood, is isolated from plasma, and serves as the genomic DNA for generation of the methylation profiles that are then correlated to the neuroinflammatory demyelinating disease.

Existing technologies do not allow for high-throughput methylation analysis in multiple genes and require substantially larger amounts of DNA (10-100 times more) for analysis, and are therefore unable to produce comprehensive methylation profiles required for diagnosis.

The present invention provides for simultaneous analysis of DNA methylation in many genes which allows for a methylation profile that is correlated to a particular disease, in this case a neuroinflammatory demyelinating disease, such as multiple sclerosis. In some embodiments, the methylation profile is based on cell free DNA from blood plasma, thereby bypassing painful and invasive sample acquisition such as lumbar puncture to obtain CSF. A methylation profile can contain any number of analyzed genes as long as the combination of genes tested is diagnostically relevant to the particular disease or other purposes. For example, FIG. 1 shows a profile of 14 genes in patients with and without MS. Since methylation profiles of neuroinflammatory demyelinating diseases are expected to be different, the method is useful for adaptation for specific localization of a neuroinflammatory demyelinating disease thereby offering a wide range of diagnostic possibilities.

Accordingly, in some embodiments, the present invention provides a method, comprising providing a biological sample from a subject (e.g., blood, plasma, serum, other bodily fluids (e.g., saliva, urine), tissue, and cytological samples), the biological sample comprising genomic DNA; detecting the presence or absence of DNA methylation in one or more genes to generate a methylation profile for the subject; and comparing the methylation profile to one or more standard methylation profiles, wherein the standard methylation profiles may comprise methylation profiles of samples that come from subjects known not to have a neuroinflammatory demyelinating disease (including prior results from the tested individual prior to a disease state) and methylation profiles of neuroinflammatory demyelinating disease samples. In certain embodiments, the detecting the presence or absence of DNA methylation comprises the digestion of the genomic DNA with a methylation-sensitive restriction enzyme followed by amplification of gene-specific DNA fragments, which optionally may include multiplex amplification. Optionally, the amplified DNA may include one or more CpG-containing sequences (or CpG islands) which are not digested by the methylation-sensitive restriction enzyme.

In further embodiments, the present invention provides a method of characterizing a neuroinflammatory demyelinating disease, comprising providing a biological sample from a subject diagnosed with a neuroinflammatory demyelinating disease, the biological sample comprising genomic DNA and detecting the presence or absence of DNA methylation in one or more genes or one or more sets of genes (e.g., each set containing 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 52, 53, 54, 55, 56, . . . genes), examples of which are listed in Table 1, thereby characterizing a neuroinflammatory demyelinating disease in the subject. In some embodiments, the methylation status of the promoter region of the gene is investigated. In some embodiments, the characterization of a neuroinflammatory demyelinating disease comprises detecting the presence or absence of multiple sclerosis. In some embodiments, the methylation profile generated allows for diagnose of multiple sclerosis in a subject.

TABLE 1

| | | Alternative | | |
|---|---|---|---|---|
| Gene | HUGO name | symbol | Alternative name | Genbank # |
| ABCB1 | ATP binding cassette, sub- | MDR1 | multidrug resistance 1 | X58723 |
| | family B, member 1 | | | |
| ACTB | actin beta | | beta actin | Y00474 |
| APAF1 | apoptotic peptidase activating | | apoptotic protease activating factor | AC013283 |
| | factor | | | |
| BRCA1 | breast cancer 1, early onset | BRCA | breast and ovarian cancer | U37574 |
| | | | susceptibility protein 1 | |
| CALCA | calcitonin/calcitonin-related | CALC | calcitonin | X15943 |
| | polypeptide, alpha | | | |
| CASP8 | caspase 8, apoptotis-related | | caspase 8 | AB038980 |
| | cysteine peptidase | | | |
| CCND2 | cyclin D2 | CYC D2 | | U47284 |
| CDH1 | cadherin 1 | | E-cadherin | L34545 |
| CDKN1A | cyclin-dependent kinase | p21waf1/cip1, | | AF497972 |
| | inhibitor 1A | p21 | | |
| CDKN1B | cyclin-dependent kinase | p27kip1 | | AB005590 |
| | inhibitor 1B | | | |
| CDKN1C | cyclin-dependent kinase | p57kip2, p57 | | D64137 |
| | inhibitor 1C | | | |
| CDKN2A | cyclin-dependent kinase | p16INK4A | | NT_037734 |
| | inhibitor 2A | | | |
| CDKN2B | cyclin-dependent kinase | p15INK4B, | | NT_037734 |
| | inhibitor 2B | p15 | | |
| DAPK1 | death associated protein kinase 1 | DAPK | death associated protein kinase | AL161787 |
| DNAJC15 | dnaJ (Hsp40) homolog, | MCJ | methylation controlled J protein | NT_024524 |
| | subfamily C, member 15 | | | |
| EDNRB | endothelin receptor type B | | | AF114163 |
| EP300 | E1A binding protein p300 | | | AL080243 |
| ESR1 promoter A | estrogen receptor 1 | ERaA | estrogen receptor alpha (proximal) | AL356311 |
| ESR1 promoter B | estrogen receptor 1 | ERaB | estrogen receptor alpha (distal) | |
| FABP3 | fatty acid binding protein 3 | MDGI | mammary derived growth inhibitor | U17081 |
| FAS | Fas (TNF receptor superfamily, | CD95 | | X87625 |
| | member 6) | | | |
| FHIT | fragile histidine triad gene | | | AF399855 |
| GPC3 | glypican 3 | | | AF003529 |
| GSTP1 | glutathione-S-transferase p1 | GSTP | | M37065 |
| HIC1 | hypermethylated in cancer 1 | HIC | | L41919 |
| ICAM1 | intercellular adhesion molecule 1 | CD54 | | M65001 |
| MCTS1 | malignant T cell amplified | MCT-1 | | AC011890 |
| | sequence | | | |
| MGMT | O-6-methylguanine DNA | | | X61657 |
| | methyltransferase | | | |
| MLH1 | mutL homolog 1 | HMLH1 | | AC011816 |
| MSH2 | mutS homolog 2 | hMSH2 | | AB006445 |
| MUC2 | mucin 2, intestinal/tracheal | | mucin 2 | U67167 |
| MYOD1 | myogenic differentiation 1 | MYF3 | myogenic factor 3 | AC124056 |
| NR3C1 | nuclear receptor subfamily 3, | GR | glucocorticoid receptor | M69074 |
| | group C, member 1 | | | |
| PAX5 | paired box gene 5 | | | AF268279 |
| PGK1 | phosphoglycerate kinase 1 | PGK | | M34017 |
| PGR distal | progesterone receptor | PR, PR-2D | progesterone receptor distal | X51730 |
| | | | promoter | |
| PGR proximal | progesterone receptor | PR, PR-1A | progesterone receptor proximal | X51730 |
| | | | promoter | |
| PLAU | plasminogen activator, | uPA | urokinase plasminogen activator | X02419 |
| | urokinase | | | |
| PRDM2 | PR domain containing 2, with | RIZ1, RIZ | retinoblastoma protein-interacting | AF472587 |
| | ZNF domain | | zinc finger protein | |
| PRKCDBP | protein kinase C, delta binding | SRBC | serum deprivation response factor | AF408198 |
| | protein | | (sdr)-related gene product that | |
| | | | binds to c-kinase | |
| PYCARD | PYD and CARD domain | TMS1 | target of methylation-induced | AF184072 |
| | containing | | silencing-1 | |
| RARB | retinoic acid receptor, beta | RAR beta 2, | retinoic acid receptor beta 2 | X56849 |
| | | RARB2, | | |
| | | RAR | | |
| RASSF1 | Ras associated (RalGDS/AF-6) | RASSF1A | | AC002481 |
| | domain family 1 | | | |
| RB1 | retinoblastoma 1 | | | AL392048 |
| RPL15 | ribosomal protein L15 | | | AB061823 |
| S100A2 | S100 calcium binding protein | S100+ | | AL162258 |
| | A2 | | | |
| SCGB3A1 | secretoglobin, family 3A, | HIN1 | high in normal-1 | AC006207 |
| | member 1 | | | |
| SFN | stratifin | | 14-3-3 sigma | AF029081 |
| SLC19A1 | solute carrier family 19 (folate | RFC1, RFC | reduced folate carrier | U92868 |
| | transporter), member 1 | | | |
| SOCS1 | suppressor of cytokine signaling 1 | SOCS | | Z46940 |
| SYK | spleen tyrosine kinase | | | AC021581 |
| TES | testis derived transcript | | | AJ250865 |
| THBS1 | thrombospondin 1 | THBS | | J04835 |
| TNFSF11 | tumor necrosis factor (ligand) | TRANCE, | osteoprotegerin ligand | AF333234 |
| | superfamily, member 11 | TRANKL, | | |
| | | OPGL | | |
| TP73 | tumor protein p73 | p73 | | AF235000 |
| VHL | von Hippel-Lindau tumor | | | AF010238 |
| | suppressor | | | |

In some embodiments, the characterization of a neuroinflammatory demyelinating disease comprises determining the risk of developing a neuroinflammatory demyelinating disease. In other embodiments, the characterization of a neuroinflammatory demyelinating disease comprises monitoring disease progression in a subject. In some embodiments, the biological sample is a plasma sample. In further embodiments, the biological sample is a biological fluid (e.g., CSF). In some embodiments, the DNA methylation comprises CpG methylation. In some preferred embodiments, detecting the presence or absence of DNA methylation comprises, for example, the digestion of said genomic DNA with a methylation-sensitive restriction enzyme followed by amplification of gene-specific DNA fragments, which optionally may include multiplex amplification. Optionally, the amplified DNA may include one or more CpG-containing sequences (or CpG islands) which are not digested by the methylation-sensitive restriction enzyme. In some embodiments, the methylation-sensitive restriction enzyme comprises Hin6I. In other embodiments the methylation sensitive restriction enzyme comprises HpaII. In certain embodiments, the neuroinflammatory demyelinating disease is multiple sclerosis, transverse myelitis, Guillain-Barré syndrome, or progressive multifocal leukoencephalopathy. However, the present invention is not limited to the method used for detecting the presence or absence of DNA methylation, indeed any method for detection of DNA methylation is contemplated for use in the methods of the present invention including, but not limited to, those found in Liu and Maekawa, 2003, Anal. Biochem. 317:259-65 and U.S. Pat. Nos. 7,144,701, 7,112,404, 7,037,650, 6,214,556 and 5,786,146, all herein incorporated by reference in their entireties.

The present invention further provides a method of diagnosing a neuroinflammatory demyelinating disease, comprising providing a biological sample from a subject, the biological sample comprising genomic DNA and detecting the presence or absence of DNA methylation in one or more genes listed in Table 1, thereby diagnosing a neuroinflammatory demyelinating disease in the subject. In some embodiments, the subject is at high risk of developing a neuroinflammatory demyelinating disease. In some embodiments, said neuroinflammatory demyelinating disease diagnosed in said subject is multiple sclerosis. In some embodiments, the subject at high risk of developing a neuroinflammatory demyelinating disease is at high risk for developing multiple sclerosis. In some embodiments, the diagnosing of multiple sclerosis comprises the identification of genetic mutations that lead to the presence or absence of DNA methylation diagnostic of multiple sclerosis. In some embodiments, the diagnosing of multiple sclerosis comprises the identification of DNA methylation of foreign nucleic acids (e.g., viral, bacterial, non-human) diagnostic of multiple sclerosis.

The present invention additionally provides a kit for characterizing a neuroinflammatory demyelinating disease, comprising reagents for (e.g., sufficient for) detecting the presence or absence of DNA methylation in one or more genes listed in Table 1. In some embodiments, the kit further comprises instructions for using the kit for characterizing a neuroinflammatory demyelinating disease in the subject. In some embodiments, the instructions comprise instructions required by the United States Food and Drug Administration for use in in vitro diagnostic products. In some embodiments, the reagents comprise reagents for digestion of the genomic DNA with a methylation-sensitive restriction enzyme followed by amplification of gene-specific DNA fragments, which optionally may include multiplex amplification. Optionally, the amplified DNA may include one or more CpG-containing sequences (or CpG islands) which are not digested by the methylation-sensitive restriction enzyme. In still further embodiments, characterizing a neuroinflammatory demyelinating disease comprises determining the risk of developing a neuroinflammatory demyelinating disease. In yet other embodiments, characterizing a neuroinflammatory demyelinating disease comprises monitoring disease progression in the subject.

In some embodiments, the present invention provides a method of characterizing or detecting a neuroinflammatory demyelinating disease, comprising providing a biological sample from a subject suspected of having a neuroinflammatory demyelinating disease or diagnosed with a neuroinflammatory demyelinating disease, the biological sample comprising genomic DNA and detecting the presence or absence of DNA methylation in one or more of the genes listed in Table 1, thereby characterizing or diagnosing a neuroinflammatory demyelinating disease in the subject.

In one embodiment, the subject is suspected of having multiple sclerosis. In some embodiments, the biological sample tested from a subject suspected of having MS is tested for the presence or absence of DNA methylation in one or more (e.g., 2, 3, 4, 5, 6, 7, 8, 9, 10, 11, 12, 13, 14) of the following genes; CASP8, ERaA, HMLH1, ICAM1, MCJ, MSH2, MYF3, P16, P57, PR-2D, RAR, RASS, RB1 and S100.

In some embodiments, the methods and compositions of the present invention can be used in conjunction with other methods for diagnosing a neuroinflammatory demyelinating disease. For example, a MS methylation profile as described herein can be used by a diagnostician in conjunction with results from a MRI of the brain or spine, results of tests run on cerebral spinal fluid, results from VEP and/or SEP analysis, presence of anti-myelin proteins, and other diagnostic tests used to diagnose MS in a patient. In some embodiments, a MS methylation profile is used to diagnose disease in a patient at an early stage wherein the aforementioned diagnostic tests would normally yield negative diagnostic test results for MS. With an earlier diagnosis than previously possible, treatment regimens can be given to a subject much sooner, thereby potentially inhibiting progression of the disease at an earlier stage than was otherwise possible with current diagnostic methods and procedures.

### BRIEF DESCRIPTION OF THE FIGURES

FIG. 1 shows the differences in methylated genes between normal blood and blood from subjects with multiple sclerosis.

### DEFINITIONS

To facilitate an understanding of the present invention, a number of terms and phrases are defined below:

As used herein, the term “subject” refers to any animal (e.g., a mammal), including, but not limited to, humans, non-human primates, rodents, and the like, which is to be the recipient of a particular treatment. Typically, the terms “subject” and “patient” are used interchangeably herein in reference to a human subject.

As used herein, the term “subject suspected of having a neuroinflammatory demyelinating disease” refers to a subject that presents one or more symptoms indicative of a neuroinflammatory demyelinating disease (e.g., brain/spinal lesions, chronic inflammation of the central nervous system, presence of anti-myelin proteins). A subject suspected of having a neuroinflammatory demyelinating disease has generally not been tested for such a disease.

As used herein, the term “providing a prognosis” refers to providing information regarding the impact of the presence of a neuroinflammatory demyelinating disease (e.g., as determined by the diagnostic methods of the present invention) on a subject's future health.

As used herein, the term “subject diagnosed with a neuroinflammatory demyelinating disease” refers to a subject having a neuroinflammatory demyelinating disease. The disease may be diagnosed using any suitable method, including but not limited to, the diagnostic methods of the present invention.

As used herein, the term “instructions for using said kit for detecting a neuroinflammatory demyelinating disease in said subject” includes instructions for using the reagents contained in the kit for the detection and characterization of the disease in a sample from a subject. In some embodiments, the instructions further comprise the statement of intended use required by the U.S. Food and Drug Administration (FDA) in labeling in vitro diagnostic products.

As used herein, the term “detecting the presence or absence of DNA methylation” refers to the detection of DNA methylation in the promoter region of one or more genes (e.g., disease markers of the present invention) of a genomic DNA sample. The detecting may be carried out using any suitable method, including, but not limited to, those disclosed herein.

As used herein, the term “monitoring disease progression in said subject” refers to the monitoring of any aspect of disease progression. In some embodiments, monitoring disease progression comprises determining the DNA methylation pattern of the subject's genomic DNA.

As used herein, the term “methylation profile” refers to a presentation of methylation status of one or more neuroinflammatory demyelinating disease marker genes in a subject's genomic DNA. In some embodiments, the methylation profile is compared to a standard methylation profile comprising a methylation profile from a known type of sample (e.g., samples known not to originate from a subject having a neuroinflammatory demyelinating disease or samples known to originate from a subject having a specific neuroinflammatory demyelinating disease). In some embodiments, methylation profiles are generated using the methods of the present invention. The profile may be presented as a graphical representation (e.g., on paper or on a computer screen), a physical representation (e.g., a gel or array) or a digital representation stored in computer memory.

As used herein, the term “non-human animals” refers to all non-human animals. Such non-human animals include, but are not limited to, vertebrates such as rodents, non-human primates, ovines, bovines, ruminants, lagomorphs, porcines, caprines, equines, canines, felines, ayes, etc.

The term “gene” refers to a nucleic acid (e.g., DNA) sequence that comprises coding sequences necessary for the production of a polypeptide, precursor, or RNA (e.g., rRNA, tRNA). The polypeptide can be encoded by a full length coding sequence or by any portion of the coding sequence so long as the desired activity or functional properties (e.g., enzymatic activity, ligand binding, signal transduction, immunogenicity, etc.) of the full-length or fragment are retained. The term also encompasses the coding region of a structural gene and the sequences located adjacent to the coding region on both the 5′ and 3′ ends for a distance of about 1 kb or more on either end such that the gene corresponds to the length of the full-length mRNA. Sequences located 5′ of the coding region and present on the mRNA are referred to as 5′ non-translated sequences. Sequences located 3′ or downstream of the coding region and present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

In addition to containing introns, genomic forms of a gene may also include sequences located on both the 5′ and 3′ end of the sequences that are present on the RNA transcript. These sequences are referred to as “flanking” sequences or regions (these flanking sequences are located 5′ or 3′ to the non-translated sequences present on the mRNA transcript). The 5′ flanking region may contain regulatory sequences such as promoters and enhancers that control or influence the transcription of the gene. The 3′ flanking region may contain sequences that direct the termination of transcription, post-transcriptional cleavage and polyadenylation.

As used herein, the terms “nucleic acid molecule encoding,” “DNA sequence encoding,” and “DNA encoding” refer to the order or sequence of deoxyribonucleotides along a strand of deoxyribonucleic acid. The order of these deoxyribonucleotides determines the order of amino acids along the polypeptide (protein) chain. The DNA sequence thus codes for the amino acid sequence.

DNA molecules are said to have “5′ ends” and “3′ ends” because mononucleotides are reacted to make oligonucleotides or polynucleotides in a manner such that the 5′ phosphate of one mononucleotide pentose ring is attached to the 3′ oxygen of its neighbor in one direction via a phosphodiester linkage. Therefore, an end of an oligonucleotide or polynucleotide is referred to as the “5′ end” if its 5′ phosphate is not linked to the 3′ oxygen of a mononucleotide pentose ring and as the “3′ end” if its 3′ oxygen is not linked to a 5′ phosphate of a subsequent mononucleotide pentose ring. As used herein, a nucleic acid sequence, even if internal to a larger oligonucleotide or polynucleotide, also may be said to have 5′ and 3′ ends. In either a linear or circular DNA molecule, discrete elements are referred to as being “upstream” or 5′ of the “downstream” or 3′ elements. This terminology reflects the fact that transcription proceeds in a 5′ to 3′ fashion along the DNA strand. The promoter and enhancer elements that direct transcription of a linked gene are generally located 5′ or upstream of the coding region. However, enhancer elements can exert their effect even when located 3′ of the promoter element or the coding region. Transcription termination and polyadenylation signals are located 3′ or downstream of the coding region.

Transcriptional control signals in eukaryotes comprise “promoter” and “enhancer” elements. Promoters and enhancers consist of short arrays of DNA sequences that interact specifically with cellular proteins involved in transcription (T. Maniatis et al., 1987, Science 236:1237). Promoter and enhancer elements have been isolated from a variety of eukaryotic sources including genes in yeast, insect and mammalian cells, and viruses (analogous control elements, i.e., promoters, are also found in prokaryote). The selection of a particular promoter and enhancer depends on what cell type is to be used to express the protein of interest. Some eukaryotic promoters and enhancers have a broad host range while others are functional in a limited subset of cell types (for review see, Voss et al., 1986, Trends Biochem. Sci., 11:287; and T. Maniatis et al., supra). Some promoter elements serve to direct gene expression in a tissue-specific manner.

As used herein, the term “promoter/enhancer” denotes a segment of DNA which contains sequences capable of providing both promoter and enhancer functions (i.e., the functions provided by a promoter element and an enhancer element, see above for a discussion of these functions). For example, the long terminal repeats of retroviruses contain both promoter and enhancer functions. The enhancer/promoter may be “endogenous” or “exogenous” or “heterologous.” An “endogenous” enhancer/promoter is one that is naturally linked with a given gene in the genome. An “exogenous” or “heterologous” enhancer/promoter is one that is placed in juxtaposition to a gene by means of genetic manipulation (i.e., molecular biological techniques such as cloning and recombination) such that transcription of that gene is directed by the linked enhancer/promoter.

The term “homology” refers to a degree of complementarity. There may be partial homology or complete homology (i.e., identity). A partially complementary sequence is a nucleic acid molecule that at least partially inhibits a completely complementary nucleic acid molecule from hybridizing to a target nucleic acid is “substantially homologous.” The inhibition of hybridization of the completely complementary sequence to the target sequence may be examined using a hybridization assay (Southern or Northern blot, solution hybridization and the like) under conditions of low stringency. A substantially homologous sequence or probe will compete for and inhibit the binding (i.e., the hybridization) of a completely homologous nucleic acid molecule to a target under conditions of low stringency. This is not to say that conditions of low stringency are such that non-specific binding is permitted; low stringency conditions require that the binding of two sequences to one another be a specific (i.e., selective) interaction. The absence of non-specific binding may be tested by the use of a second target that is substantially non-complementary (e.g., less than about 30% identity); in the absence of non-specific binding the probe will not hybridize to the second non-complementary target.

When used in reference to a double-stranded nucleic acid sequence such as a cDNA or genomic clone, the term “substantially homologous” refers to any probe that can hybridize to either or both strands of the double-stranded nucleic acid sequence under conditions of low stringency as described above.

When used in reference to a single-stranded nucleic acid sequence, the term “substantially homologous” refers to any probe that can hybridize (i.e., it is the complement of) the single-stranded nucleic acid sequence under conditions of low stringency as described above.

As used herein, the term “hybridization” is used in reference to the pairing of complementary nucleic acids. Hybridization and the strength of hybridization (i.e., the strength of the association between the nucleic acids) is impacted by such factors as the degree of complementary between the nucleic acids, stringency of the conditions involved, the Tₘ of the formed hybrid, and the G:C ratio within the nucleic acids. A single molecule that contains pairing of complementary nucleic acids within its structure is said to be “self-hybridized.”

As used herein, the term “Tₘ” is used in reference to the “melting temperature.” The melting temperature is the temperature at which a population of double-stranded nucleic acid molecules becomes half dissociated into single strands. The equation for calculating the Tₘ of nucleic acids is well known in the art. As indicated by standard references, a simple estimate of the Tₘ value may be calculated by the equation: Tₘ=81.5+0.41(% G+C), when a nucleic acid is in aqueous solution at 1 M NaCl (See e.g., Anderson and Young, Quantitative Filter Hybridization, in Nucleic Acid Hybridization (1985)). Other references include more sophisticated computations that take structural as well as sequence characteristics into account for the calculation of Tₘ.

As used herein the term “stringency” is used in reference to the conditions of temperature, ionic strength, and the presence of other compounds such as organic solvents, under which nucleic acid hybridizations are conducted. With “high stringency” conditions, nucleic acid base pairing will occur only between nucleic acid fragments that have a high frequency of complementary base sequences. Thus, conditions of “weak” or “low” stringency are often required with nucleic acids that are derived from organisms that are genetically diverse, as the frequency of complementary sequences is usually less.

“High stringency conditions” when used in reference to nucleic acid hybridization comprise conditions equivalent to binding or hybridization at 42° C. in a solution consisting of 5×SSPE (43.8 g/l NaCl, 6.9 g/l NaH₂PO₄.H₂O and 1.85 g/l EDTA, pH adjusted to 7.4 with NaOH), 0.5% SDS, 5× Denhardt's reagent and 100 vg/ml denatured salmon sperm DNA followed by washing in a solution comprising 0.1×SSPE, 1.0% SDS at 42° C. when a probe of about 500 nucleotides in length is employed.

“Medium stringency conditions” when used in reference to nucleic acid hybridization comprise conditions equivalent to binding or hybridization at 42° C. in a solution consisting of 5×SSPE (43.8 g/l NaCl, 6.9 g/l NaH₂PO₄.H₂O and 1.85 g/l EDTA, pH adjusted to 7.4 with NaOH), 0.5% SDS, 5× Denhardt's reagent and 100 μg/ml denatured salmon sperm DNA followed by washing in a solution comprising 1.0×SSPE, 1.0% SDS at 42° C. when a probe of about 500 nucleotides in length is employed.

“Low stringency conditions” comprise conditions equivalent to binding or hybridization at 42° C. in a solution consisting of 5×SSPE (43.8 g/l NaCl, 6.9 g/l NaH₂PO₄.H₂O and 1.85 g/l EDTA, pH adjusted to 7.4 with NaOH), 0.1% SDS, 5× Denhardt's reagent (50× Denhardt's contains per 500 ml: 5 g Ficoll (Type 400, Pharamcia), 5 g BSA (Fraction V; Sigma)) and 100 μg/ml denatured salmon sperm DNA followed by washing in a solution comprising 5×SSPE, 0.1% SDS at 42° C. when a probe of about 500 nucleotides in length is employed.

The art knows well that numerous equivalent conditions may be employed to comprise low stringency conditions; factors such as the length and nature (DNA, RNA, base composition) of the probe and nature of the target (DNA, RNA, base composition, present in solution or immobilized, etc.) and the concentration of the salts and other components (e.g., the presence or absence of formamide, dextran sulfate, polyethylene glycol) are considered and the hybridization solution may be varied to generate conditions of low stringency hybridization different from, but equivalent to, the above listed conditions. In addition, the art knows conditions that promote hybridization under conditions of high stringency (e.g., increasing the temperature of the hybridization and/or wash steps, the use of formamide in the hybridization solution, etc.) (see definition above for “stringency”).

“Amplification” is a special case of nucleic acid replication involving template specificity. It is to be contrasted with non-specific template replication (i.e., replication that is template-dependent but not dependent on a specific template). Template specificity is here distinguished from fidelity of replication (i.e., synthesis of the proper polynucleotide sequence) and nucleotide (ribo- or deoxyribo-) specificity. Template specificity is frequently described in terms of “target” specificity. Target sequences are “targets” in the sense that they are thought to be sorted out from other nucleic acid. Amplification techniques have been designed primarily for this sorting out.

Template specificity is achieved in most amplification techniques by the choice of enzyme. Taq and Pfu polymerases, by virtue of their ability to function at high temperature, are found to display high specificity for the sequences bounded and thus defined by the primers; the high temperature results in thermodynamic conditions that favor primer hybridization with the target sequences and not hybridization with non-target sequences (H. A. Erlich (ed.), PCR Technology, Stockton Press (1989)).

As used herein, the term “amplifiable nucleic acid” is used in reference to nucleic acids that may be amplified by any amplification method. It is contemplated that “amplifiable nucleic acid” will usually comprise “sample template.”

As used herein, the term “sample template” refers to nucleic acid originating from a sample that is analyzed for the presence of “target”. In contrast, “background template” is used in reference to nucleic acid other than sample template that may or may not be present in a sample. Background template is most often inadvertent. It may be the result of carryover, or it may be due to the presence of nucleic acid contaminants thought to be purified away from the sample. For example, nucleic acids from organisms other than those to be detected may be present as background in a test sample.

As used herein, the term “primer” refers to an oligonucleotide, whether occurring naturally as in a purified restriction digest or produced synthetically, that is capable of acting as a point of initiation of synthesis when placed under conditions in which synthesis of a primer extension product that is complementary to a nucleic acid strand is induced, (i.e., in the presence of nucleotides and an inducing agent such as DNA polymerase and at a suitable temperature and pH). The primer is preferably single stranded for maximum efficiency in amplification, but may alternatively be double stranded. If double stranded, the primer is first treated to separate its strands before being used to prepare extension products. Preferably, the primer is an oligodeoxyribonucleotide. The primer must be sufficiently long to prime the synthesis of extension products in the presence of the inducing agent. The exact lengths of the primers will depend on many factors, including temperature, source of primer and the use of the method.

As used herein, the term “probe” refers to an oligonucleotide (i.e., a sequence of nucleotides), whether occurring naturally as in a purified restriction digest or produced synthetically, recombinantly or by PCR amplification, that is capable of hybridizing to another oligonucleotide of interest. A probe may be single-stranded or double-stranded. Probes are useful in the detection, identification and isolation of particular gene sequences. It is contemplated that any probe used in the present invention will be labeled with any “reporter molecule,” so that is detectable in any detection system, including, but not limited to enzyme (e.g., ELISA, as well as enzyme-based histochemical assays), fluorescent, radioactive, and luminescent systems. It is not intended that the present invention be limited to any particular detection system or label.

As used herein, the terms “PCR product,” “PCR fragment,” and “amplification product” refer to the resultant mixture of compounds after two or more cycles of the PCR steps of denaturation, annealing and extension are complete. These terms encompass the case where there has been amplification of one or more segments of one or more target sequences.

As used herein, the term “amplification reagents” refers to those reagents (deoxyribonucleotide triphosphates, buffer, etc.), needed for amplification except for primers, nucleic acid template and the amplification enzyme. Typically, amplification reagents along with other reaction components are placed and contained in a reaction vessel (test tube, microwell, etc.).

As used herein, the terms “restriction endonucleases” and “restriction enzymes” refer to bacterial enzymes, each of which cut double-stranded DNA at or near a specific nucleotide sequence.

The terms “in operable combination,” “in operable order,” and “operably linked” as used herein refer to the linkage of nucleic acid sequences in such a manner that a nucleic acid molecule capable of directing the transcription of a given gene and/or the synthesis of a desired protein molecule is produced. The term also refers to the linkage of amino acid sequences in such a manner so that a functional protein is produced.

The term “isolated” when used in relation to a nucleic acid, as in “an isolated oligonucleotide” or “isolated polynucleotide” refers to a nucleic acid sequence that is identified and separated from at least one component or contaminant with which it is ordinarily associated in its natural source. Isolated nucleic acid is such present in a form or setting that is different from that in which it is found in nature. In contrast, non-isolated nucleic acids as nucleic acids such as DNA and RNA found in the state they exist in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs that encode a multitude of proteins. However, isolated nucleic acid encoding a given protein includes, by way of example, such nucleic acid in cells ordinarily expressing the given protein where the nucleic acid is in a chromosomal location different from that of natural cells, or is otherwise flanked by a different nucleic acid sequence than that found in nature. The isolated nucleic acid, oligonucleotide, or polynucleotide may be present in single-stranded or double-stranded form. When an isolated nucleic acid, oligonucleotide or polynucleotide is to be utilized to express a protein, the oligonucleotide or polynucleotide will contain at a minimum the sense or coding strand (i.e., the oligonucleotide or polynucleotide may be single-stranded), but may contain both the sense and anti-sense strands (i.e., the oligonucleotide or polynucleotide may be double-stranded).

As used herein, the term “in vitro” refers to an artificial environment and to processes or reactions that occur within an artificial environment. In vitro environments can consist of, but are not limited to, test tubes and cell culture. The term “in vivo” refers to the natural environment (e.g., an animal or a cell) and to processes or reactions that occur within a natural environment.

As used herein, the term “sample” is used in its broadest sense. In one sense, it is meant to include a specimen or culture obtained from any source, as well as biological and environmental samples. Biological samples may be obtained from animals (including humans) and encompass fluids, solids, tissues, and gases. Biological samples include blood products, such as plasma, serum and the like. Environmental samples include environmental material such as surface matter, soil, water, crystals and industrial samples. Such examples are not however to be construed as limiting the sample types applicable to the present invention.

### DETAILED DESCRIPTION

Advances in molecular biology are making an impact on the design and development of new, more efficient drugs, and more precise diagnostic procedures. However, there is still a noticeable gap when a given approach is already well established and widely used for research goals, but its clinical applications remain unrecognized and its usefulness for diagnostic and prognostic purposes remains untested.

Microarray-based expression profiling has emerged as a very powerful approach for broad evaluation of gene expression in various systems. However, this approach has its limitations, and one of the most important is the requirement of a certain minimal amount of mRNA: if it is below a certain level due to low promoter activity, short half-life of mRNA, or small amounts of starting material expression of the gene cannot be unambiguously detected. An additional concern is the stability of RNA, which in many cases is difficult to control such that the absence of a signal for a certain gene might reflect artificially introduced degradation rather than genuine decrease in expression.

DNA is a much more stable milieu for analysis, and DNA methylation in regions with increased density of CpG dinucleotides (e.g., CpG islands) has been shown to correlate inversely with corresponding gene expression when such CpG islands are located in the promoter and/or the first exon of the gene. A number of techniques have been developed for methylation analysis and arguably the most popular of them—methylation-specific PCR or MSP—takes advantage of modification of unmethylated cytosines by bisulfite and alkali which results in their conversion to uracils, changing their partners from guanosine to thymidine. This change can be detected by PCR with primers that contain appropriate substitutions. A substantial amount of data on gene-specific methylation has been acquired using MSP.

The present invention improves methylation analysis by providing a technique for high throughput analysis without losses in the sensitivity. The first phase of the assay involves digestion of genomic DNA with a methylation-sensitive enzyme (e.g., HpaII or Hin6I), which cuts unmethylated sites, for example GCGC, while leaving even hemi-methylated sites intact. Efficiency of this step determines the discriminating power of the approach, since the next procedure—amplification of the CpG island-containing fragments with primers flanking the methylation specific restriction enzyme site—serves mainly to increase the sensitivity of the assay. Reference is made to U.S. application Ser. No. 10/677,701, entitled “Methylation Profile of Cancer,” which was filed on Oct. 2, 2003, and claims the benefit of U.S. provisional application No. 60/415,628, filed on Oct. 2, 2002, the contents of which are incorporated herein by reference in their entireties. Reference also is made to U.S. application Ser. No. 11/872,956, entitled “Methylation Profile of Cancer,” which was filed on Oct. 16, 2007, and claims the benefit of U.S. provisional application No. 60/852,360, filed on Oct. 17, 2007, the contents of which are incorporated herein by reference in their entireties.

The present invention overcomes many of the problems of mRNA arrays (e.g., stability of RNA and quantitation of expression) by evaluating gene expression by measuring methylation profiles of CpG-containing sequences. Regions of unusually high GC content have been described in many genes (Cooper et al., 1983, DNA 2:131) and may be referred to as “CpG islands”; the cytosine of CpG islands can be modified by methyltransferase to produce a methylated derivative—5-methylcytosine (Cooper et al., supra; Baylin et al., 1992, AIDS Res Hum Retroviruses 8:811). If a methylated cytosine is located in the promoter region of a gene, it is likely to be silenced (Cooper et al., supra). Silencing of various tumor suppressor and growth regulator genes (Rountree et al., 2001, Oncogene 20: 3156; Yang et al., 2001, Endocr. Relat. Cancer 8: 115-127) has been linked to cancer development and progression in general (Baylin et al., supra; Jones, 1986, Cancer Res. 46:461). Accordingly, in some embodiments, the present invention provides diagnostics comprising the identification of methylation patterns in samples from subjects suspected of having a neuroinflammatory demyelinating disease such as multiple sclerosis. None of the known genes is methylated in all cases of the disease, thus simultaneous analysis of several genes within the same sample increases the clinical value of the assay. Testing need not provide a definitive diagnostic result. The provision of data that demonstrates an increased risk finds use in both medical and research settings. The methods also find use for a variety of research applications.

In some embodiments, the present invention provides methylation-based procedures for neuroinflammatory demyelinating disease detection. The present invention demonstrates that microarray-mediated methylation assay (M³A) can achieve high sensitivity and high specificity. Importantly, M³A performance does not require subjective evaluation of assay data, making its results observer-independent.

M³A was used for methylation detection. A limited number of GCGC sites in each gene is evaluated by this approach (Melnikov et al., 2005, Nucl. Acids Res. 33:e93), so in some embodiments, choosing a different set of sites within the same set of genes can affect the final readout. Accordingly, in some embodiments, a variety of sets of sites within the same set of genes is utilized. This feature of the assay indicates that, in some embodiments, assignment of “methylated” or “unmethylated” values depends on the selection of the GCGC sites within each region.

Signal detection in M³A is based in part on competitive hybridization of two PCR products (one from digested and the second from undigested DNA of the same sample), which are labeled with different fluorophores, so that hybridization results are scored as fluorescence intensity for each of them. Assignment of “methylated” (M) and “unmethylated” (UM) calls depends on the ratio of fluorescence of undigested and digested DNA, which, in preferred embodiments, produce one of two values; 1, if the fragment is methylated and digestion does not affect its representation, and infinity, if the fragment is unmethylated and no signal from digested DNA is detected. This type of ideal distribution is rarely seen even in cell lines because of intrinsic heterogeneity of biological material (Melnikov et al., 2005, supra).

Additional complications may be associated with the unequal performance of fluorophores Cy3 and Cy5, which ideally should not influence signal distribution, but in reality can affect the results. To adjust results, a “self-self” hybridization is sometimes used for expression microarrays when aliquots of the same DNA sample are labeled separately with Cy3 and Cy5 fluorescent dyes and co-hybridized to the same microarray. Thus, in some embodiments, a similar adjustment is done for methylation detection, so the Cy5/Cy3 ratio from two identical aliquots can be used as the threshold of methylated fragments. Using this approach it is possible to convert numerical data of microarray experiments to binary readout defining methylated and unmethylated calls. However, the present invention is not limited to the method used for detecting the presence or absence of DNA methylation, indeed it is contemplated that any method that detects the presence or absence of DNA methylation finds utility in the present invention.

In some embodiments, the present invention provides methods of correlating methylation patterns with clinical outcomes. In other embodiments, the present invention provides methods of disease monitoring during treatment and rapid screening of a high-risk population.

Differential methylation of CpG-containing sequences provides an alternative way to characterize expression—or more accurately, repression—profiles of cell lines and tissues. Repression of heavily methylated genes is thought to depend on interactions of methylated cytosines with MeCP2, which either interferes with transcriptional complex assembly or prevents its movement.

Experiments conducted during the course of development of the present invention provide a novel methylation assay designed to provide a fast estimate on the methylation status of chosen genes. The assay uses restriction endonuclease specificity to discriminate between methylated and unmethylated sequences, and on PCR reaction to amplify surviving templates. The present invention is not limited to the use of methylation specific restriction enzymes and PCR. Any method that examines methylation state (e.g., by selective cleavage, modification, etc.) followed by detection, is contemplated by the present invention. The number and specifics of the genes analyzed can be altered based on the choice of primers.

The methods of the present invention are amenable to detection of differences in expression profiles when inadequate quantities of starting material are available. In some embodiments, the method includes extensive digestion of genomic DNA with a methylation-sensitive restriction enzyme (e.g., HpaII or Hin6I), followed by amplification of gene-specific DNA fragments, which optionally may include multiplex amplification. Optionally, the amplified DNA may include one or more CpG-containing sequences (or CpG islands) which are not digested by the methylation-sensitive restriction enzyme.

The markers of the present invention, when used to characterize or diagnose a neuroinflammatory demyelinating disease, may be detected by any appropriate methodology or technology, including any future developed technologies that identify differentially methylated DNA sequences.

### EXPERIMENTAL

The following examples are provided in order to demonstrate and further illustrate certain preferred embodiments and aspects of the present invention and are not to be construed as limiting the scope thereof.

### Example 1

### General Experimental Outline

Purified genomic DNA from plasma samples is divided into two parts; one of the samples is treated with the methylation-sensitive restriction enzyme Hin6I while the other one is used as a control. Both control and digested DNA is used as templates for nested PCR with aminoallyl-dUTP added at the second round of amplification. Following amplification, the incorporated aminoallyl-dUTP is coupled to reactive Cy5 or Cy3 dyes, creating fluorescently labeled probes. One of the dyes is used for PCR products from undigested control DNA, while another is used for PCR products from Hin6I-digested DNA. Both labeled products are mixed together and applied to a custom-designed microarray slide for competitive hybridization. A microarray reader is used to quantify fluorescence of each fluorophore in every spot of the array, and the Cy5/Cy3 ratio used to assess methylation status. Methylated fragments produce Cy5/Cy3 ratios close to 1, while unmethylated fragments have ratios higher than 1. Statistical analysis of hybridization data is performed to identify informative features and build the classifier for each disease marker panel.

### Example 2

### Restriction Enzyme Digestion of Tissues

Exhaustive digestion of DNA is done with the methylation sensitive restriction endonuclease Hin6I (Fermentas International, Inc., recognition site GCGC). Successful digestion of 4 ng of DNA is done with 40 U of the enzyme in 100 μl of reaction mix at 37° C. for 48 hr. To exclude non-specific degradation of DNA during a long incubation we use the second aliquot of DNA incubated without the enzyme. This control is then processed side-by-side with digested DNA and only fragments with an adequate signal from control DNA are scored. After digestion is completed, the DNA is purified and quantitated as previously described.

### Example 3

### PCR Amplification of Sample DNA

The first round of PCR amplification, nested PCR, is performed using 400 pg of digested and control DNAs. Empirically assembled primer groups for multiplex reactions allow simultaneous amplification of five targets in each reaction. Final concentration of primers is 0.2 μM for each of the multiplex PCR reactions. KlenTaq®1 (DNA Polymerase Technology, Inc) is used at 20 Upper 50 μl reaction. To PCR buffer supplied with the enzyme we add betaine (Sigma) to 1.5M and dNTPs (Sigma) to 0.25 mM. The tubes are placed into a preheated ABI 9600 thermocycler and incubated for 5 min prior to addition of KlenTaq® 1. PCR is started for 25 cycles by initial denaturation at 95° C. followed by 25 cycles of; 45 sec-62° C.; 1 min-72° C.; 1 min cycling conditions. After 25 cycles the PCR reactions are kept at 4° C.

The PCR products of the first round are purified using QIAquick® PCR Purification Kit (Qiagen) and quantified. Amplification products for corresponding DNAs are combined, and 400 pg are used for the second PCR, which is assembled as above except for dNTPs, where a mix of aminoallyl-dUTP (Biotium, Inc,) and dTTP (3:1) is used. The second round of PCR is performed as the first except only 20 cycles are used. PCR products are purified using QIAquick® PCR Purification Kit and products are combined.

The second PCR products are dried in vacuum and dissolved in 5 μl of 200 mM NaHCO₃ buffer (pH 9.0). Cy3 or Cy5 fluorescent dyes in DMSO are added to each tube, mixed and spun. Labeling continues for two hours at room temperature in the dark. Unreacted Cy dyes are quenched by 4.5 μl 4M hydroxylamine for 15 minutes in the dark. Final purification is done by precipitating labeled PCR products with ethanol.

### Example 4

### Development and Manufacture of the Array

Oligonucleotide arrays are custom designed by Microarrays, Inc (Nashville, Tenn.). Probes for the array are 50-60 mers to keep hybridization and washing temperatures high (Relogio et al., 2002, Nucleic Acids Res 30:e51). Probes have been designed according to the Affymetrix model (Mei et al., 2003, Proc. Natl. Acad. Sci. 10:11237-11242). Controls may be present on the array, for example: (1) transcribed regions from Arabidopsis thaliana (definitive negative control, heterologous); (2) transcribed regions of human α-tubulin, β-actin and glyceraldehyde-phosphate-dehydrogenase (GAPDH, definitive negative controls, homologous); (3) promoters of β-actin, phosphoglycerate kinase (PGK1) and/or ribosomal protein L15 (conditional homologous negative control). HPLC-purified oligonucleotides with an amino group and a six-carbon spacer at the 5′-end are spotted on aminosilane-modified glass slides in triplicate, so each slide contains three identical subarrays. Attachment of the probe is done by incubation at 60° C. for 3.5 hr and for 10 min at 120° C. Slides are stored under vacuum in the dark at room temperature. Genes to be tested in the DNA methylation assay include those listed in Table 1 that are specific to the method being performed. These genes represent different functional groups; all of them have been identified as methylated in different disease states.

### Example 5

### Probe Hybridizations with Microarray

Competitive hybridization of the PCR probes to oligonucleotide arrays is done in rotating tubes in the hybridization chamber. The slides are pre-hybridized for 1 hr at 42° C. in 5×SSC, 0.1% SDS, 1% BSA, rinsed with deionized water and dried by short centrifugation. Hybridization space is created on the slide by Microarray GeneFrames (AbGene, Rochester, N.Y.). Denatured DNA is added to the array, the coverslip is sealed, and the slides are incubated in the dark at 42° C. for 18 hr. After hybridization the GeneFrame and the coverslip are removed, and the slides are washed with shaking in a set of buffers heated to 42° C.: 5 min in 1×SSC, 0.1% SDS; 5 min in 0.1×SSC, 0.1% SDS; 3 min in 0.1×SSC, 0.1% SDS. Slides are dried by a short, low-speed centrifugation and stored in the dark before scanning.

During optimization of the procedure, a single PCR product was labeled with two different fluorophores, probes were mixed, and used for hybridization. In this mixture Cy5- and Cy3-labeled fragments were represented equally imitating conditions for methylated fragments. Mean Cy5/Cy3 ratio calculated from such experiments produced the normalization coefficient to account for fluorophore-related differences in labeling and detection.

### Example 6

### Signal Detection and Sample Scoring

Scanning is done with ScanArray™ 4000XL (Packard BioChip) according to the manual. ScanArray™ software allows selection of different Photo Multiplier Tube (PMT) gain parameters to adjust to different quantum yields of Cy3 and Cy5 fluorophores; these parameters were established experimentally based on the maximum signal strength and minimum background/PMT noise. The protocol (EasyScan) for detection of two fluorophore hybridizations is used.

Quantitation of the signal is done using the Adaptive Circle algorithm of the ScanArray™ software. Initially the signals are normalized to account for differences in fluorophore incorporation and detection. The percentage of the signal for an individual spot relative to the total signal from the corresponding fluorophore is used to normalize signals across the array and then the ratio of the Cy5/Cy3 percentages for each spot is computed. An alternative technique makes use of the expected distribution of the ratios and allows for differences in methylation status at the majority of sites under investigation. Suppose we observe (xᵢ,yᵢ), i=1, . . . , n where xᵢ is the Cy3 intensity and yᵢ is the Cy5 intensity for specimen i. The goal of normalization is to find a function, ƒ(.) such that yᵢ≧ƒ(xᵢ), for most of the regions. A smoothed lower boundary for the cloud (xᵢ,yᵢ), i=1, . . . , n can be achieved by non-parametric quantile regression in which the 10-20% quantile curve is used as the normalizing function ƒ(.). Such a function will allow measurement error so that some yᵢ values may be slightly less than ƒ(xᵢ). In the end, the ratio rᵢ=yᵢ/ƒ(xᵢ) is then used to measure the signal. This technique will produce ratios that are either close to 1 or >1 and will reduce the number of methylation sites with middle range ratios (1.3 to 2). After the signals are normalized, ratios will be computed.

The percentage normalization method allows the detection of very high Cy3:Cy5 ratios (up to 5,000) and approximately equal ratios (between 0.8 and 1.2), which correspond to unmethylated and methylated sites, respectively. Some genes fall in the intermediate range (genes methylated in some part of the population with ratios between 1.3 and 2) and are removed from the diagnostic set. The quantile regression normalization method eliminates these intermediate values, so no manual adjustment is required.

The pattern of expression microarray analysis is followed and non-specific filtering is applied to remove uninvolved or uninformative features from consideration before selecting the most divergent in their methylation status (Scholtens and von Heydebreck, 2005, Studies is Bioinformatics and Computational Biology Solutions using R and Bioconductor, Gentleman et al., Eds.). Two non-specific filters are applied: 1) for all samples investigated, 80% of the samples must give interpretable ratios (<1.3 or >2); and 2) at least 10% differential methylation must be observed across all samples (e.g., 90% methylated and 10% unmethylated). After the non-specific filtering step, methylation sites (features) are selected on the basis of differential status in the test and control tissues. For feature selection and classifier design the Support Vector Machine algorithm is used, which has been developed for pattern recognition tasks (Model et al., 2001, Bioinformatics 17(Suppl. 1):S157-164). All samples are divided into a training set and a test set. Initially, Support Vector Machine is used with the training set to select features and create the classifier function, which is then validated with a “leave-one-out” analysis using the same training set (Lee et al., 2004, IEEE Trans. Neural. Netw. 15:750-757). Results are subsequently evaluated using the Fisher's Exact test.

### Results

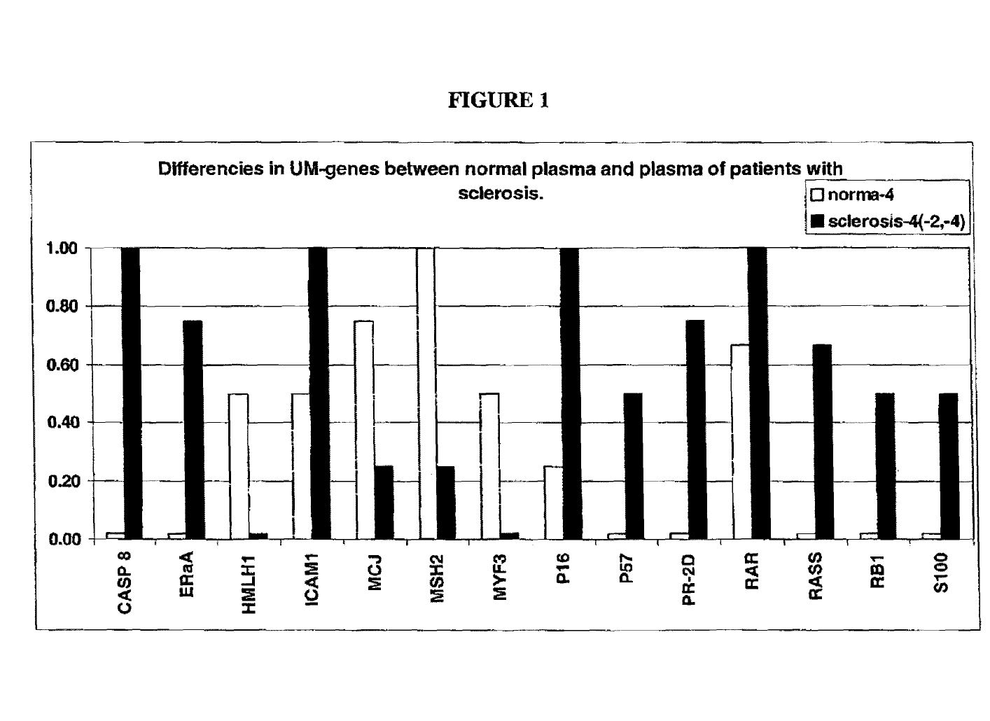

Multiple sclerosis methylation profiling is seen in FIG. 1. Genes studied include CASP8, ERaA, HMLH1, ICAM1, MCJ, MSH2, MYF3, P16, P57, PR-2D, RAR, RASS, RB1 and S100. The graph demonstrates the ratio of unmethylated genes relative to the methylation status of their normal counterpart. The genes demonstrating decreased methylation in multiple sclerosis as compared to a patient without multiple sclerosis include CASP8, ERaA, ICAM1, P16, P57, PR-2D, RAR, RASS, RB1 and S100, whereas the converse is true with the genes HMLH1, MCJ, MSH2 and MYF3. FIG. 1 shows distinctive gene methylation patterns for multiple sclerosis, thereby allowing for profiling, diagnosing, and characterization of this disease.

All publications and patents mentioned in the above specification are herein incorporated by reference. Various modifications and variations of the described compositions and methods of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the present invention.

1. A method for characterizing a sample from a subject, comprising:
a) providing a sample from said subject, wherein said sample comprises nucleic acid;
b) exposing said sample to reagents for detecting methylation status; and
c) determining the methylation status of the promoter of two or more genes from the group consisting of caspase 8, estrogen receptor 1, mutL homolog 1, intercellular adhesion molecule 1, methylation controlled J protein, mutS homolog 2, myogenic differentiation 1, cyclin-dependent kinase inhibitor 2A, cyclin-dependent kinase inhibitor 1C, progesterone receptor, retinoic acid receptor, Ras associated domain family 1, retinoblastoma 1 and S100 calcium binding protein.

2. A method of characterizing a neuroinflammatory demyelinating disease, comprising:
a) providing a sample from a subject, said sample comprising genomic DNA; and
b) detecting the presence or absence of DNA methylation in five or more genes from the group consisting of caspase 8, estrogen receptor 1, mutL homolog 1, intercellular adhesion molecule 1, methylation controlled J protein, mutS homolog 2, myogenic differentiation 1, cyclin-dependent kinase inhibitor 2A, cyclin-dependent kinase inhibitor 1C, progesterone receptor, retinoic acid receptor, Ras associated domain family 1, retinoblastoma 1 and S100 calcium binding protein, thereby characterizing a neuroinflammatory demyelinating disease, in said subject.

3. The method of claim 1, wherein said detecting a neuroinflammatory demyelinating disease comprises detecting the presence or absence of multiple sclerosis.

4. The method of claim 1, wherein said sample is plasma.

5. The method of claim 2, wherein said DNA methylation comprises CpG methylation.

6. The method of claim 2, wherein said neuroinflammatory demyelinating disease is multiple sclerosis.

7. The method of claim 2, wherein said sample is plasma.

8. The method of claim 2, wherein at least one of said genes used is upregulated and wherein one of said genes used is down-regulated.

9.-10. (canceled)

11. A method for diagnosing a neuroinflammatory demyelinating disease in a subject, comprising:
(a) reacting isolated genomic DNA from the subject and a methylation-sensitive restriction enzyme; wherein the genomic DNA comprises a plurality of promoters from different genes, and the enzyme cleaves unmethylated CpG sequences in the promoters and does not cleave methylated CpG sequences in the promoters;
(b) contacting the genomic DNA thus reacted and a plurality of pairs of specific primers in an amplification mixture, the pairs of specific primers being configured to hybridize to the genomic DNA and to amplify a plurality of different promoters through a region comprising an uncleaved CpG sequence;
(c) reacting the amplification mixture;
(d) detecting one or more amplified promoters in the reacted amplification mixture or the absence thereof, thereby diagnosing the neuroinflammatory demyelinating disease.

12. The method of claim 11, wherein the plurality of pairs of specific primers comprises at least two pairs of specific primers and each of the two pairs of specific primers is configured to amplify a different gene selected from the group consisting of caspase 8, estrogen receptor 1, mutL homolog 1, intercellular adhesion molecule 1, methylation controlled J protein, mutS homolog 2, myogenic differentiation 1, cyclin-dependent kinase inhibitor 2A, cyclin-dependent kinase inhibitor 1C, progesterone receptor, retinoic acid receptor, Ras associated domain family 1, retinoblastoma 1 and S100 calcium binding protein.

13. The method of claim 11, wherein the plurality of pairs of specific primers comprises at least five pairs of specific primers and each of the five pairs of specific primers is configured to amplify a different gene selected from the group consisting of caspase 8, estrogen receptor 1, mutL homolog 1, intercellular adhesion molecule 1, methylation controlled J protein, mutS homolog 2, myogenic differentiation 1, cyclin-dependent kinase inhibitor 2A, cyclin-dependent kinase inhibitor 1C, progesterone receptor, retinoic acid receptor, Ras associated domain family 1, retinoblastoma 1 and S100 calcium binding protein.

14. The method of claim 11, wherein the genomic DNA is isolated from blood or plasma.

15. The method of claim 11, wherein the neuroinflammatory demyelinating disease is multiple sclerosis.

16. The method of claim 11, wherein detecting one or more amplified promoters in the reacted amplification mixture or the absence thereof comprises:
(1) contacting a microarray and the reacted amplification mixture, the microarray comprising a plurality of DNA samples, each of which hybridizes to one of the plurality of different promoters; and
(2) detecting hybridization or the lack of hybridization between DNA in the reacted amplification mixture and one or more of the plurality of DNA samples of the microarray thereby obtaining a methylation profile.

17. The method of claim 11, further comprising comparing the methylation profile for the subject and a standard methylation profile selected from the group consisting of a standard methylation profile for normal subjects, a standard methylation profile for subjects having the neuroinflammatory demyelinating disease, and both standard methylation profiles.

18. The method of claim 11, further comprising the step of separating the isolated genomic DNA of step (a) into: (i) a control sample and (ii) an experimental sample and adding control nucleic acid to both the control and experimental samples, wherein the control nucleic acid comprises at least one known CpG sequence that is unmethylated.

19. The method of claim 18, wherein the control sample is not reacted with the methylation-sensitive restriction enzyme and the experimental sample is reacted with the methylation-sensitive restriction enzyme, and wherein both the control and experimental samples are contacted with primers for the control nucleic acid under conditions such that a fragment of the control nucleic acid is amplified if the known CpG sequence is uncleaved.
